(19) United States
(12) Patent Application Publication
(10) Pub. No.: US 2013/0101630 A1
(43) Pub. Date: Apr. 25, 2013

(54) HIGHLY PURE VARENICLINE OR A PHARMACEUTICALLY ACCEPTABLE SALT THEREOF SUBSTANTIALLY FREE OF METHYLVARENICLINE IMPURITY

(71) Applicants: Nitin Sharadchandra Pradhan, Maharashtra (IN); Arvapally Seshu Kumar, Andhra Pradesh (IN); Nikhil Trivedi, Maharashtra (IN); Krishnadatt Sharma, Mumbai (IN)

(72) Inventors: Nitin Sharadchandra Pradhan, Maharashtra (IN); Arvapally Seshu Kumar, Andhra Pradesh (IN); Nikhil Trivedi, Maharashtra (IN); Krishnadatt Sharma, Mumbai (IN)

(73) Assignee: ACTAVIS GROUP PTC EHF, Hafnarfjordur (IS)

(21) Appl. No.: 13/581,491

(22) PCT Filed: Mar. 8, 2011
(86) PCT No.: PCT/IB11/00983

§ 371 Date: Dec. 12, 2012

(30) Foreign Application Priority Data
Mar. 9, 2010 (IN) 611/CHE/2010

(51) Int. Cl. C07D 471/08; A61K 31/4985; C07C 59/255

(52) U.S. Cl. CPC C07D 471/08; C07C 59/255; A61K 31/4985
USPC 424/400; 544/343; 514/250

(57) ABSTRACT

Provided herein is an impurity of varenicline, methylvarenicline, 6-methyl-5,8,14-triazatetracyclo[10.3.1.0^{2,11},0^{4,9}]hexadeca-2(11),3,5,7,9-pentaene, and a process for the preparation and isolation thereof. Provided further herein is a highly pure varenicline or a pharmaceutically acceptable salt thereof substantially free of methylvarenicline impurity, processes for the preparation thereof, and pharmaceutical compositions comprising highly pure varenicline or a pharmaceutically acceptable salt thereof substantially free of methylvarenicline impurity.

### CROSS REFERENCE TO RELATED APPLICATION

This application claims the benefit of priority to Indian provisional application No. 611/CHE/2010, filed on Mar. 9, 2010, which is incorporated herein by reference in its entirety.

### FIELD OF THE DISCLOSURE

Disclosed herein is an impurity of varenicline, methylvarenicline impurity, and a process for the preparation and isolation thereof. Disclosed further herein is a highly pure varenicline or a pharmaceutically acceptable salt thereof substantially free of methylvarenicline impurity, a process for the preparation thereof, and pharmaceutical compositions comprising highly pure varenicline or a pharmaceutically acceptable salt thereof substantially free of methylvarenicline impurity.

### BACKGROUND

Varenicline, 5,8,14-triazatetracyclo[10.3.1.0^{2,11},0^{4,9}]hexadeca-2(11),3,5,7,9-pentaene, is known to bind to neuronal nicotinic acetylcholine specific receptor sites and is useful in modulating cholinergic function. This compound is useful in the treatment of inflammatory bowel disease, irritable bowel syndrome, spastic dystonia, chronic pain, acute pain, vasoconstriction, anxiety, panic disorder, depression, cognitive dysfunction, drug/toxin-induced cognitive impairment (e.g., from alcohol, barbiturates, vitamin deficiencies, recreational drugs, lead, arsenic, mercury), particularly, nicotine dependency, addiction and withdrawal; including use in smoking cessation therapy. Varenicline is represented by the following structural formula:

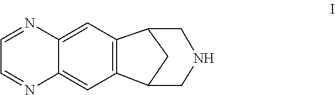 [SMILES: c1cnc2cc3c(cc2n1)C1CNCC3C1]

and its first synthesis was disclosed in U.S. Pat. No. 6,410,550 (hereinafter referred to as the '550 patent). Varenicline is sold by Pfizer under the brand name CHANTIX™ for the treatment of α₄β₂ nicotinic acetylcholine receptor subtypes. It is orally administered as tablets containing 0.85 mg or 1.71 mg of varenicline tartrate equivalent to 0.5 mg or 1 mg of varenicline.

The '550 patent describes various processes for the preparation of aryl fused azapolycyclic compounds, which includes varenicline, and their pharmaceutically acceptable salts, combinations with other therapeutic agents, and methods of using such combinations in the treatment of neurogical and psychological disorders. Varenicline has been exemplified as a free base and a hydrochloride salt in the '550 patent.

U.S. Pat. No. 6,890,927 (hereinafter referred to as the '927 patent) discloses tartrate salts, including L-tartrate, D-tartrate, D,L-tartrate and meso-tartrate, of varenicline and their polymorphs, processes for their preparation, and pharmaceutical compositions thereof. The '927 patent further discloses various polymorphs of the varenicline L-tartrate salt, including two anhydrous polymorphs (Forms A & B) and a hydrate polymorph (Form C), and characterizes them by powder X-ray diffraction (P-XRD), X-ray crystal structure, solid state ¹³C NMR spectroscopy, and Differential Scanning calorimetry (DSC).

Varenicline tartrate, 7,8,9,10-tetrahydro-6,10-methano-6H-pyrazino[2,3-h][3]benzazepine, (2R,3R)-2,3-dihydroxybutanedioate (1:1), has a molecular weight of 361.35 Daltons, and a molecular formula of C₁₃H₁₃N₃.C₄H₆O₆. Varenicline tartrate is represented by the following structural formula:

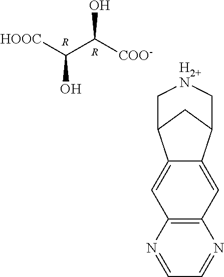

U.S. Pat. Nos. 6,897,310 and 6,951,938 describe a process for the preparation of aryl fused azapolycyclic compounds and their pharmaceutically acceptable salts in combination with another therapeutic agents and methods of using such combinations in the treatment of neurogical and psychological disorder. The '938 patent discloses the ring closure for making quinoxalines by reacting a diamine compound with glyoxal or glyoxal derivatives in water or other polar solvents such as tetrahydrofuran, dimethylformamide or dimethylsulfoxide at a temperature of about 40° C. to about 100° C.

PCT publication No. WO 2004/108725 describes a process for the preparation of substituted quinoxalines by cyclization of the corresponding diamine compound with 2,3-dihydroxy-1,4-dioxane.

The synthetic routes described in the above mentioned prior art suffer from disadvantages such as high cost of reagents, the use of pyrophoric and hazardous reagents, the use of additional reagents and low yields of product. Hence, these routes are not advisable for scale up operations.

PCT Publication No. WO 2008/060487 (hereinafter referred to as the '487 application) discloses crystal forms of intermediates used in the process for the preparation of varenicline tartrate, including the varenicline free base. According to the '487 application, the varenicline free base exists in four crystalline forms (Form A, Form C, Form D and Form E).

PCT Publication No. WO 2010/023561 (hereinafter referred to as the '561 application), filed by the present applicant, discloses an improved and convenient process for the preparation of varenicline or a pharmaceutically acceptable salt thereof by reacting a protected diaminoazatricyclo compound with a haloacetaldehyde compound, optionally in the presence of an oxygen source, to provide a protected triazatetracyclo compound, which is then deprotected to produce varenicline.

It is known that synthetic compounds can contain extraneous compounds or impurities resulting from their synthesis or degradation. The impurities can be unreacted starting materials, by-products of the reaction, products of side reactions, or degradation products. Generally, impurities in an active pharmaceutical ingredient (API) may arise from degradation of the API itself, or during the preparation of the API. Impurities in varenicline or any active pharmaceutical ingredient (API) are undesirable and might be harmful.

Regulatory authorities worldwide require that drug manufacturers isolate, identify and characterize the impurities in their products. Furthermore, it is required to control the levels of these impurities in the final drug compound obtained by the manufacturing process and to ensure that the impurity is present in the lowest possible levels, even if structural determination is not possible.

The product mixture of a chemical reaction is rarely a single compound with sufficient purity to comply with pharmaceutical standards. Side products and byproducts of the reaction and adjunct reagents used in the reaction will, in most cases, also be present in the product mixture. At certain stages during processing of the active pharmaceutical ingredient, the product is analyzed for purity, typically, by HPLC, TLC or GC analysis, to determine if it is suitable for continued processing and, ultimately, for use in a pharmaceutical product. Purity standards are set with the intention of ensuring that an API is as free of impurities as possible, and, thus, are as safe as possible for clinical use. The United States Food and Drug Administration guidelines recommend that the amounts of some impurities are limited to less than 0.1 percent.

Generally, impurities are identified spectroscopically and by other physical methods, and then the impurities are associated with a peak position in a chromatogram (or a spot on a TLC plate). Thereafter, the impurity can be identified by its position in the chromatogram, which is conventionally measured in minutes between injection of the sample on the column and elution of the particular component through the detector, known as the “retention time” (“Rt”). This time period varies daily based upon the condition of the instrumentation and many other factors. To mitigate the effect that such variations have upon accurate identification of an impurity, practitioners use “relative retention time” (“RRt”) to identify impurities. The RRt of an impurity is its retention time divided by the retention time of a reference marker.

It is known by those skilled in the art, the management of process impurities is greatly enhanced by understanding their chemical structures and synthetic pathways, and by identifying the parameters that influence the amount of impurities in the final product.

There is a need for highly pure varenicline or a pharmaceutically acceptable salt thereof substantially free of impurities, as well as processes for the preparation thereof.

### SUMMARY

In one aspect, provided herein is an isolated methylvarenicline compound, 6-methyl-5,8,14-triazatetracyclo[10.3.1.0^{2,11},0^{4,9}]hexadeca-2(11),3,5,7,9-pentaene, having the following structural formula A:

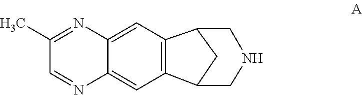 [SMILES: Cc1cnc2cc3c(cc2n1)C1CNCC3C1]

or a pharmaceutically acceptable acid addition salt thereof. The compound of formula A is also referred to herein as the methylvarenicline impurity.

In another aspect, encompassed herein is a process for synthesizing and isolating the methylvarenicline compound of formula A.

In another aspect, provided herein is a highly pure varenicline or a pharmaceutically acceptable salt thereof substantially free of methylvarenicline impurity.

In yet another aspect, encompassed herein is a process for preparing the highly pure varenicline or a pharmaceutically acceptable salt thereof substantially free of methylvarenicline impurity.

In another aspect, provided herein is a pharmaceutical composition comprising highly pure varenicline or a pharmaceutically acceptable salt thereof substantially free of methylvarenicline impurity, and one or more pharmaceutically acceptable excipients.

In still another aspect, provided herein is a pharmaceutical composition comprising highly pure varenicline or a pharmaceutically acceptable salt thereof substantially free of methylvarenicline impurity made by the process disclosed herein, and one or more pharmaceutically acceptable excipients.

In still further aspect, encompassed is a process for preparing a pharmaceutical formulation comprising combining highly pure varenicline or a pharmaceutically acceptable salt thereof substantially free of methylvarenicline impurity with one or more pharmaceutically acceptable excipients.

In another aspect, the highly pure varenicline or a pharmaceutically acceptable salt thereof substantially free of methylvarenicline impurity disclosed herein for use in the pharmaceutical compositions has a D₉₀ particle size of less than or equal to about 300 microns, specifically about 1 micron to about 200 microns, and most specifically about 10 microns to about 100 microns.

### DETAILED DESCRIPTION

According to one aspect, there is provided a methylvarenicline compound, 6-methyl-5,8,14-triazatetracyclo[10.3.1.0^{2,11},0^{4,9}]hexadeca-2(11),3,5,7,9-pentaene, having the following structural formula A:

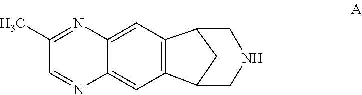 [SMILES: Cc1cnc2cc3c(cc2n1)C1CNCC3C1]

or a pharmaceutically acceptable acid addition salt thereof.

The pharmaceutically acceptable acid addition salts of methylvarenicline can be derived from a therapeutically acceptable acid such as hydrochloric acid, hydrobromic acid, sulfuric acid, nitric acid, phosphoric acid, acetic acid, propionic acid, oxalic acid, succinic acid, maleic acid, fumaric acid, benzenesulfonic acid, toluenesulfonic acid, citric acid, and tartaric acid.

Specific pharmaceutically acceptable acid addition salts of methylvarenicline are hydrochloride, hydrobromide, oxalate, sulphate, fumarate, succinate, maleate, besylate, tosylate, tartrate; and more specifically the tartrate salt.

According to another aspect, there is provided an impurity of varenicline, the methylvarenicline impurity, 6-methyl-5,8,14-triazatetracyclo[10.3.1.0^{2,11},0^{4,9}]hexadeca-2(11),3,5,7,9-pentaene, of formula A.

The methylvarenicline impurity has been identified, isolated and synthesized. The methylvarenicline impurity was detected and resolved from varenicline by HPLC with an RRt of 1.6. The structure of the compound of formula A was deduced with the aid of ¹H, ¹³C NMR and IR spectroscopy and FAB mass spectrometry. The parent ion at 226 is consistent with the assigned structure.

The methylvarenicline disclosed herein is characterized by data selected from a ¹H NMR (400 MHz, CDCl₃) δ (ppm): 2.08-2.11 (d, 1H), 2.48-2.51 (m, 1H), 2.88-2.91 (d, 2H), 3.12-3.16 (d, 2H), 3.23 (s, 2H), 7.75-7.80 (d, 2H), 8.66 (s, 1H); MS: EI⁺ m/z (MH+): 226.3; and IR spectra on KBr having absorption bands at about 3270, 2849-2943, 1460, 1164, 859, 690 and 797 cm⁻¹.

According to another aspect, there is provided an isolated methylvarenicline impurity. Methylvarenicline impurity formed during the synthesis of varenicline or a pharmaceutically acceptable salt thereof can be isolated by subjecting the varenicline or a pharmaceutically acceptable salt thereof that contains the methylvarenicline impurity to column chromatography. The column chromatography comprises using a silica gel, as a stationary phase, and a gradient of eluents that remove methylvarenicline impurity from the column on which it adsorbed.

In one embodiment, the methylvarenicline of formula A is prepared according to the process exemplified in the Example 2 as disclosed herein.

Regarding the specific RRt value of the methylvarenicline impurity disclosed herein, it is well known to a person skilled in the art that the RRt values may vary from sample to sample due to, inter alia, instrument errors (both instrument to instrument variation and the calibration of an individual instrument) and differences in sample preparation. Thus, it has been generally accepted by those skilled in the art that independent measurement of an identical RRt value can differ by amounts of up to ±0.02.

Thus there is a need for a method for determining the level of impurities in varenicline samples and removing the impurities.

Extensive experimentation was carried out by the present inventors to reduce the level of the methylvarenicline impurity in varenicline. As a result, it has been found that the methylvarenicline impurity formed in the preparation of the varenicline can be reduced or substantially completely removed by the process disclosed herein.

According to another aspect, there is provided a highly pure varenicline or a pharmaceutically acceptable salt thereof substantially free of methylvarenicline impurity.

As used herein, “highly pure varenicline or a pharmaceutically acceptable salt thereof substantially free of methylvarenicline impurity” refers to varenicline or a pharmaceutically acceptable salt thereof comprising the methylvarenicline impurity in an amount of less than about 0.15 area-% as measured by HPLC. Specifically, the varenicline, as disclosed herein, contains less than about 0.1 area-%, more specifically less than about 0.05 area-%, still more specifically less than about 0.02 area-% of the methylvarenicline impurity, and most specifically is essentially free of the methylvarenicline impurity.

In one embodiment, the highly pure varenicline or a pharmaceutically acceptable salt thereof disclosed herein comprises the methylvarenicline impurity in an amount of about 0.01 area-% to about 0.1 area-%, specifically in an amount of about 0.01 area-% to about 0.05 area-%, as measured by HPLC.

In another embodiment, the highly pure varenicline or a pharmaceutically acceptable salt thereof disclosed herein has a purity of greater than about 99%, specifically greater than about 99.5%, more specifically greater than about 99.9%, and most specifically greater than about 99.95% as measured by HPLC. For example, the purity of the highly pure varenicline or a pharmaceutically acceptable salt thereof is about 99% to about 99.95%, or about 99.5% to about 99.99%.

In yet another embodiment, the highly pure varenicline or a pharmaceutically acceptable salt thereof disclosed herein is essentially free of the methylvarenicline impurity.

The term “varenicline or a pharmaceutically acceptable salt thereof essentially free of methylvarenicline impurity” refers to varenicline or a pharmaceutically acceptable salt thereof contains a non-detectable amount of the methylvarenicline impurity as measured by HPLC.

Specific pharmaceutically acceptable salts of varenicline include, but are not limited to, hydrochloride, hydrobromide, sulphate, phosphate, tartrate, fumarate, maleate, oxalate, acetate, propionate, succinate, mandelate, mesylate, besylate and tosylate; and a more specific salt is varenicline tartrate.

According to another aspect, there is provided a process for preparing highly pure varenicline of formula I:

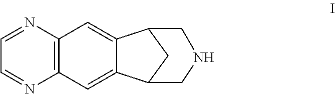 [SMILES: c1cnc2cc3c(cc2n1)C1CNCC3C1]

or a pharmaceutically acceptable salt thereof substantially free of methylvarenicline impurity, comprising:
a) reacting a protected diaminoazatricyclo compound of formula III:

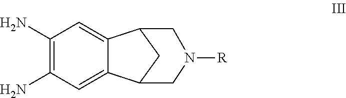

wherein ‘R’ represents a nitrogen protecting group, with a haloacetaldehyde compound of formula IV:

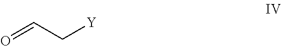

wherein ‘Y’ represents a halogen atom selected from the group consisting of F, Cl, Br and I; optionally in the presence of an oxygen source, to produce a crude protected triazatetracyclo compound of formula II:

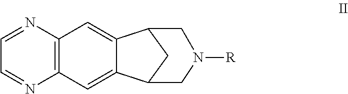

wherein R is as defined in formula III; and
b) recrystallizing the crude protected triazatetracyclo compound of formula II obtained in step-(a) from a first solvent to produce a pure protected triazatetracyclo compound of formula II;
c) deprotecting the pure compound of formula II obtained in step-(b) to produce a reaction mass containing varenicline free base;
d) recovering the varenicline free base as a residue from the reaction mass obtained in step-(c);
e) dissolving or suspending the varenicline free base obtained in step-(d) in a solvent medium comprising a second solvent and a third solvent to produce a solution or suspension, wherein the second solvent is an alcohol solvent and wherein the third solvent is an ether solvent; and
f) isolating and/or recovering highly pure varenicline free base substantially free of methylvarenicline impurity from the solution or suspension obtained in step-(e), and optionally converting the varenicline obtained in to a pharmaceutically acceptable salt thereof.

In one embodiment, the reaction in step-(a) is carried out in the presence of a solvent. The term solvent also includes mixtures of solvents.

Exemplary solvents employed in step-(a) include, but are not limited to, water, an alcohol, a chlorinated hydrocarbon, a ketone, a polar aprotic solvent, a nitrile, an ester, and mixtures thereof.

Specifically, the solvent is selected from the group consisting of water, methanol, ethanol, isopropyl alcohol, n-propanol, tert-butanol, n-butanol, methylene chloride, ethyl dichloride, chloroform, carbon tetrachloride, acetone, methyl isobutyl ketone, N,N-dimethylformamide, N,N-dimethylacetamide, dimethylsulfoxide, acetonitrile, propionitrile, ethyl acetate, isopropyl acetate, and mixtures thereof; and more specifically, the solvent is selected from the group consisting of water, dimethylsulfoxide, N,N-dimethylformamide, dimethylacetamide, and mixtures thereof.

In one embodiment, the amount of aprotic solvent employed in the coupling reaction can range from about 5 volumes to about 25 volumes, and specifically from about 7 volumes to about 15 volumes with respect to the diaminoazatricyclo compound of formula III.

Exemplary oxygen sources employed in step-(a) include, but are not limited to, lead monoxide, manganese dioxide, mercuric iodide, ceric ammonium nitrate, and the like. A specific oxygen source is lead monoxide.

In one embodiment, the condensation reaction in step-(a) is carried out at a temperature of about 0° C. to the reflux temperature of the solvent used, specifically at a temperature of about 25° C. to the reflux temperature of the solvent used for at least 1 hour, and most specifically at the reflux temperature of the solvent used for about 2 hours to about 10 hours. The reaction mass may be quenched with water after completion of the reaction.

As used herein, “reflux temperature” means the temperature at which the solvent or solvent system refluxes or boils at atmospheric pressure.

Exemplary nitrogen protecting group ‘R’ in the compounds of formulae II and III include, but is not limited to, acetyl, trifluoroacetyl, trichloroacetyl, pyrrolidinylmethyl, cumyl, benzhydryl, trityl, benzyloxycarbonyl (Cbz), 9-fluorenylmethyloxy carbonyl (Fmoc), benzyloxymethyl (BOM), pivaloyloxymethyl (POM), trichloroethxoycarbonyl (Troc), 1-adamantyloxycarbonyl (Adoc), allyl, allyloxycarbonyl, trimethylsilyl, tert.-butyldimethylsilyl, triethylsilyl (TES), triisopropylsilyl, trimethylsilylethoxymethyl (SEM), t-butoxycarbonyl (BOC), t-butyl, 1-methyl-1,1-dimethylbenzyl, pyrridinyl and pivaloyl. Specific nitrogen protecting groups are trifluoroacetyl, trichloroacetyl, trichloroethxoycarbonyl, benzyloxycarbonyl, t-butoxycarbonyl, allyloxycarbonyl and pivaloyl. A most specific nitrogen protecting group is trifluoroacetyl.

In one embodiment, the halogen atom ‘Y’ in the compound of formula IV is Cl.

The reaction mass containing the crude protected triazatetracyclo compound of formula II obtained in step-(a) is subjected to usual work up such as a washing, an extraction, a layer separation, an evaporation, a filtration, a pH adjustment, or a combination thereof.

Exemplary first solvents used in step-(b) include, but are not limited to, water, an alcohol, a ketone, a nitrile, and mixtures thereof. The term solvent also includes mixtures of solvents.

In one embodiment, the first solvent is selected from the group consisting of water, methanol, ethanol, n-propanol, isopropanol, n-butanol, isobutanol, tert-butanol, amyl alcohol, acetone, methyl ethyl ketone, methyl isobutyl ketone, methyl tert-butyl ketone, acetonitrile, and mixtures thereof; more specifically, the first solvent is selected from the group consisting of water, methanol, ethanol, isopropanol, n-butanol, and mixtures thereof; and most specifically isopropanol.

The recrystallization in step-(b) is carried out by dissolving the crude triazatetracyclo compound of formula II in the first solvent to form a clear solution, and crystallizing the pure triazatetracyclo compound of formula II from the solution by forcible or spontaneous crystallization.

In one embodiment, the crude triazatetracyclo compound of formula II is dissolved in the first solvent at a temperature of about 30° C. to the reflux temperature of the solvent used, specifically at about 40° C. to the reflux temperature of the solvent used, and most specifically at the reflux temperature of the solvent used.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by adding an anti-solvent to the solution or a combination thereof.

The term “anti-solvent” refers to a solvent which when added to an existing solution of a substance reduces the solubility of the substance.

Exemplary anti-solvents include, but are not limited to, an ether, a hydrocarbon solvent, and mixtures thereof.

In one embodiment, the anti-solvent is selected from the group consisting of diisopropyl ether, diethyl ether, tetrahydrofuran, dioxane, n-pentane, n-hexane and n-heptane and their isomers, cyclohexane, toluene, xylene, and mixtures thereof. Specific anti-solvents are diisopropyl ether, diethyl ether and mixtures thereof.

In another embodiment, the crystallization is carried out by cooling the solution while stirring at a temperature of below 30° C., specifically at a temperature of about 0° C. to about 30° C., and most specifically at about 20° C. to about 30° C.

The pure protected triazatetracyclo compound of formula II obtained in step-(b) is recovered by methods such as filtration, filtration under vacuum, decantation, centrifugation, or a combination thereof. In one embodiment, the pure protected triazatetracyclo compound of formula II is recovered by filtration employing a filtration media of, for example, a silica gel or celite.

The removal of protecting groups in step-(c) can be achieved by conventional methods used in peptide chemistry and are described e.g. in the relevant chapters of standard reference works such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981.

In one embodiment, the deprotection in step-(c) is carried out by treating the protected triazatetracyclo compound of formula II with a base in a reaction inert solvent.

The base used for deprotection is an organic or inorganic base. Specific organic bases are triethyl amine, trimethylamine, N,N-diisopropylethylamine, N-methylmorpholine and N-methylpiperidine. Specific inorganic bases are ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide. A most specific base is sodium hydroxide or potassium hydroxide.

Exemplary reaction inert solvents used for deprotection in step-(c) include, but are not limited to, water, an alcohol, a chlorinated hydrocarbon, a ketone, a polar aprotic solvent, a nitrile, an ester, and mixtures thereof. In one embodiment, the solvent is selected from the group consisting of water, methanol, ethanol, isopropyl alcohol, acetone, and mixtures thereof.

The reaction mass containing the varenicline of formula I obtained in step-(c) may be subjected to usual work up such as a washing, an extraction, a charcoal treatment, a layer separation, an evaporation, a filtration, a pH adjustment, or a combination thereof.

In one embodiment, the second solvent used in step-(e) is selected from the group consisting of methanol, ethanol, n-propanol, isopropanol, n-butanol, isobutanol, tert-butanol, amyl alcohol, and mixtures thereof; and the third solvent used in step-(e) is selected from the group consisting of tetrahydrofuran, 2-methyltetrahydrofuran, dioxane, diethyl ether, diisopropyl ether, monoglyme, diglyme, and mixtures thereof.

Specifically, the solvent medium used in step-(e) is a mixture of methanol and diisopropyl ether.

In one embodiment, the varenicline free base in step-(e) is dissolved in the solvent medium at a temperature of about 30° C. to about the reflux temperature of the solvent medium used, specifically at about 40° C. to the reflux temperature of the solvent medium used, and most specifically at the reflux temperature of the solvent medium used.

In another embodiment, the suspension in step-(e) is prepared by suspending the varenicline free base in the solvent medium while stirring at a temperature of about 0° C. to about the reflux temperature of the solvent medium used. In one embodiment, the suspension is stirred at a temperature of about 40° C. to about the reflux temperature of the solvent medium used for at least 30 minutes, and more specifically at about 45° C. to about 80° C. for about 1 hour to about 10 hours.

The solution obtained in step-(e) is optionally subjected to carbon treatment or silica gel treatment. The carbon treatment or silica gel treatment is carried out by methods known in the art, for example by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate containing varenicline free base by removing charcoal or silica gel. Specifically, the finely powdered carbon is an active carbon. A specific mesh size of silica gel is 40-500 mesh, and more specifically 60-120 mesh.

The isolation and recovery of highly pure varenicline free base substantially free of methylvarenicline impurity in step-(f) is carried out by the methods as described above.

In one embodiment, the isolation is carried out by cooling the solution or suspension while stirring at a temperature of below 30° C. for at least 30 minutes, specifically at about 0° C. to about 30° C. for about 1 hour to about 20 hours, and more specifically at about 20° C. to about 30° C. for about 2 hours to about 10 hours.

Pharmaceutically acceptable salts of varenicline can be prepared in high purity by using the highly pure varenicline substantially free of methylvarenicline impurity obtained by the methods disclosed herein, by known methods.

Specific pharmaceutically acceptable salts of varenicline are obtained from organic and inorganic acids include hydrochloric acid, hydrobromic acid, sulfuric acid, phosphoric acid, tartaric acid, derivatives of tartaric acid, fumaric acid, maleic acid, oxalic acid, acetic acid, propionic acid, succinic acid, mandelic acid, citric acid; and a most specific salt being varenicline tartrate.

The pure varenicline or a pharmaceutically acceptable salt thereof obtained by the process disclosed herein may be further dried in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

The varenicline of formula I or a pharmaceutically acceptable salt thereof obtained by the process disclosed herein has a purity (measured by High Performance Liquid Chromatography, hereinafter referred to as ‘HPLC’) greater than about 99%, specifically greater than about 99.5%, and more specifically greater than about 99.9%. For example, the purity of the varenicline or a pharmaceutically acceptable salt thereof can be about 99% to about 99.95%, or about 99.5% to about 99.99%.

According to another aspect, there is provided a process for synthesizing and isolating the methylvarenicline compound, 6-methyl-5,8,14-triazatetracyclo[10.3.1.0^{2,11},0^{4,9}]hexadeca-2(11),3,5,7,9-pentaene, of formula A:

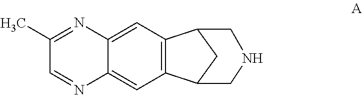 [SMILES: Cc1cnc2cc3c(cc2n1)C1CNCC3C1]

or a pharmaceutically acceptable acid addition salt thereof, comprising:
a) reacting a protected diaminoazatricyclo compound of formula III:

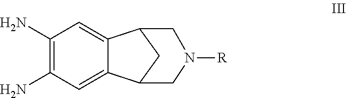

wherein ‘R’ represents a nitrogen protecting group, with a haloacetone compound of formula VI:

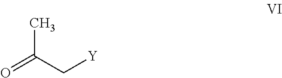

wherein ‘Y’ represents a halogen atom selected from the group consisting of F, Cl, Br and I; optionally in the presence of an oxygen source, to provide a protected triazatetracyclo compound of formula V:

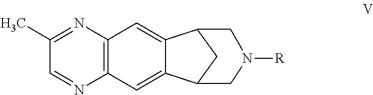

wherein R is as defined in formula III; and
b) deprotecting the compound of formula V to produce a reaction mass containing methylvarenicline of formula A; and
c) isolating and/or recovering methylvarenicline of formula A from the reaction mass obtained in step-(b) and optionally converting the methylvarenicline obtained in to a pharmaceutically acceptable acid addition salt thereof.

In one embodiment, the reaction in step-(a) is carried out in the presence of a solvent. The term solvent also includes mixtures of solvents.

Exemplary solvents employed in step-(a) include, but are not limited to, water, an alcohol, a chlorinated hydrocarbon, a ketone, a polar aprotic solvent, a nitrile, an ester, and mixtures thereof.

Specifically, the solvent is selected from the group consisting of water, methanol, ethanol, isopropyl alcohol, n-propanol, tert-butanol, n-butanol, methylene chloride, ethyl dichloride, chloroform, carbon tetrachloride, acetone, methyl isobutyl ketone, N,N-dimethylformamide, N,N-dimethylacetamide, dimethylsulfoxide, acetonitrile, propionitrile, ethyl acetate, isopropyl acetate, and mixtures thereof. More specifically, the solvent is selected from the group consisting of water, dimethylsulfoxide, N,N-dimethylformamide, dimethylacetamide, and mixtures thereof.

In one embodiment, the amount of solvent employed in the coupling reaction is about 5 volumes to about 25 volumes, and specifically about 7 volumes to about 15 volumes with respect to the diaminoazatricyclo compound of formula III.

In one embodiment, the oxygen source employed in step-(a) is selected from the group as described above. A specific oxygen source is lead monoxide.

In another embodiment, the condensation reaction in step-(a) is carried out at a temperature of about 0° C. to the reflux temperature of the solvent used for at least 1 hour, specifically at a temperature of about 25° C. to 100° C. for about 2 hours to about 20 hours, and most specifically at about 40° C. to 80° C. for about 3 hours to about 15 hours. The reaction mass may be quenched with water after completion of the reaction.

The nitrogen protecting group ‘R’ in the compounds of formulae III and V is selected from the group as described above. A most specific nitrogen protecting group is trifluoroacetyl.

In one embodiment, the halogen atom ‘Y’ in the compound of formula VI is Cl.

The reaction mass containing the protected triazatetracyclo compound of formula V obtained in step-(a) is optionally subjected to usual work up methods as described above. The reaction mass may be used directly in the next step to produce methylvarenicline of formula A, or the compound of formula V may be isolated and then used in the next step.

In one embodiment, the compound of formula V is isolated from a suitable solvent by methods such as cooling, seeding, partial removal of the solvent from the solution, by adding an anti-solvent to the solution, evaporation, vacuum distillation, or a combination thereof.

In one embodiment, the deprotection in step-(b) is carried out by treating the protected triazatetracyclo compound of formula V with a base in a reaction inert solvent.

The base used for deprotection is an organic or inorganic base selected from the group as described above. Specific inorganic bases are ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide. A most specific base is sodium hydroxide or potassium hydroxide.

The reaction inert solvent used for deprotection in step-(b) is selected from the group as described above. In one embodiment, the solvent is selected from the group consisting of water, methanol, ethanol, isopropyl alcohol, acetone, and mixtures thereof.

The reaction mass containing the methylvarenicline of formula I obtained in step-(b) is optionally subjected to usual work up methods as described above.

The isolation of methylvarenicline in step-(c) is carried out using a suitable solvent by conventional methods such as cooling, seeding, partial removal of the solvent from the solution, by adding an anti-solvent to the solution, evaporation, vacuum distillation, or a combination thereof.

In one embodiment, the solvent used for isolating the methylvarenicline is selected from the group consisting of water, methanol, ethanol, isopropyl alcohol, t-butanol, acetone, dichloromethane, tetrahydrofuran, dioxane, diethyl ether, diisopropyl ether, monoglyme, diglyme, and mixtures thereof. A most specific solvent is diisopropyl ether.

In another embodiment, the isolation in step-(c) is carried out by cooling the solution while stirring at a temperature of below 30° C. for at least 30 minutes, specifically at about 0° C. to about 30° C. for about 1 hour to about 20 hours, and more specifically at about 0° C. to about 25° C. for about 2 hours to about 10 hours.

The recovery of methylvarenicline of formula A in step-(c) is accomplished by the methods as described above.

The methylvarenicline of formula A obtained by the process disclosed herein has a purity (measured by High Performance Liquid Chromatography, hereinafter referred to as ‘HPLC’) greater than about 98%, specifically greater than about 98.5%, more specifically greater than about 99%, and still more specifically greater than about 99.9%.

Pharmaceutically acceptable salts of methylvarenicline can be prepared in high purity by using the substantially pure methylvarenicline free base obtained by the methods disclosed herein, by known methods.

Further encompassed herein is the use of the highly pure varenicline or a pharmaceutically acceptable salt thereof substantially free of methylvarenicline impurity for the manufacture of a pharmaceutical composition together with a pharmaceutically acceptable carrier.

A specific pharmaceutical composition of highly pure varenicline or a pharmaceutically acceptable salt thereof substantially free of methylvarenicline impurity is selected from a solid dosage form and an oral suspension.

In one embodiment, the highly pure varenicline or a pharmaceutically acceptable salt thereof substantially free of methylvarenicline impurity has a D₉₀ particle size of less than or equal to about 300 microns, specifically about 1 micron to about 200 microns, and most specifically about 10 microns to about 100 microns.

In another embodiment, the particle sizes of the highly pure varenicline or a pharmaceutically acceptable salt thereof substantially free of methylvarenicline impurity are produced by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided a method for treating a patient suffering from inflammatory bowel disease, irritable bowel syndrome, spastic dystonia, chronic pain, acute pain, vasoconstriction, anxiety, panic disorder, depression, cognitive dysfunction and drug/toxin-induced cognitive impairment, comprising administering a therapeutically effective amount of the highly pure varenicline or a pharmaceutically acceptable salt thereof substantially free of methylvarenicline impurity, or a pharmaceutical composition that comprises a therapeutically effective amount of highly pure varenicline or a pharmaceutically acceptable salt thereof substantially free of methylvarenicline impurity along with pharmaceutically acceptable excipients.

According to another aspect, there are provided pharmaceutical compositions comprising highly pure varenicline or a pharmaceutically acceptable salt thereof substantially free of methylvarenicline impurity prepared according to the processes disclosed herein and one or more pharmaceutically acceptable excipients.

According to another aspect, there is provided a process for preparing a pharmaceutical formulation comprising combining highly pure varenicline or a pharmaceutically acceptable salt thereof substantially free of methylvarenicline impurity prepared according to processes disclosed herein, with one or more pharmaceutically acceptable excipients.

Yet in another embodiment, pharmaceutical compositions comprise at least a therapeutically effective amount of highly pure varenicline or a pharmaceutically acceptable salt thereof substantially free of methylvarenicline impurity. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The highly pure varenicline or a pharmaceutically acceptable salt thereof substantially free of methylvarenicline impurity may also be administered as suppositories, ophthalmic ointments and suspensions, and parenteral suspensions, which are administered by other routes.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In one embodiment, capsule dosage forms contain highly pure varenicline or a pharmaceutically acceptable salt thereof substantially free of methylvarenicline impurity within a capsule which may be coated with gelatin. Tablets and powders may also be coated with an enteric coating. Suitable enteric coating agents include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### Experimental Details:

HPLC method for measuring chemical purity:

The purity was measured by high performance liquid chromatography under the following conditions:

### Column: Acclaim Polar Advantage-II C18 (150×4.6) mm, 5.0μ

Make: Dionex; (Product No. 063197)
   Detector wavelength: UV at 210 nm
   Flow rate: 0.80 ml/min
   Injection volume: 10.0 μl
   Run time: 70 minutes
   Oven temperature: 40° C.
   Diluent: 0.10% Ortho phosphoric acid:Methanol (85:15) (v/v)

### Elution: Gradient

The following examples are given for the purpose of illustrating the present disclosure and should not be considered as limitation on the scope or spirit of the disclosure.

### EXAMPLES

### Example 1

### Preparation of pure 5,8,14-Triazatetracyclo[10.3.1.0^{2,11},0^{4,9}]hexadeca-2(11),3,5,7,9-pentaene (Varenicline)

### Step-I: Preparation of Crude 1-(5,8,14-Triazatetracyclo[10.3.1.0^{2,11}.0^{4,9}]hexadeca-2(11),3,5,7,9-pentaene)-2,2,2-trifluoro-ethanone

1-(4,5-Diamino-10-aza-tricyclo[6.3.1.0^{2,7}]dodeca-2(7),3,5-trien-10-yl)-2,2,2-trifluoro-ethanone (100 gm), 50% chloroacetaldehyde solution in water (27.5 g) and lead monoxide (78.2 g) were added to 20% aqueous dimethyl sulfoxide (80:20, dimethyl sulfoxide:water) (1700 ml), and the mixture was heated at 55-60° C. The reaction mixture was stirred at 55-60° C. for 30 minutes, followed by the addition of 50% chloroacetaldehyde solution in water (13.75 g) at 55-60° C. The reaction mixture was stirred for 30 minutes at 55-60° C., followed by the addition of 50% chloroacetaldehyde solution in water (13.75 g) at 55-60° C. The reaction mixture was stirred for 6-10 hours at 55-60° C., the reaction mass was cooled to 25-30° C., followed by the addition of water (1700 ml). The unreacted lead monoxide was filtered and washed with water (1700 ml). The filtrate was extracted with methylene chloride (5×1000 ml) at 25-30° C. The organic layers were combined and washed with 1 N hydrochloric acid (3×1000 ml). The methylene chloride layer was concentrated under vacuum at below 45° C. and then degassed for 30 minutes at 45° C. The residue was dissolved in isopropanol (300 ml) at reflux temperature, followed by cooling the clear solution at 25-30° C., and stirring the resulting suspension at 25-30° C. for 3 hours. The separated solid was filtered and washed with isopropanol (2×50 ml) to yield 38 g of crude 1-(5,8,14-triazatetracyclo[10.3.1.0^{2,11}.0^{4,9}]hexadeca-2(11),3,5,7,9-pentaene)-2,2,2-trifluoro-ethanone (Purity by HPLC: 99.5%; Content of 1-(6-Methyl-5,8,14-triazatetracyclo[10.3.1.0^{2,11}.0^{4,9}]hexadeca-2(11),3,5,7,9-pentaene)-2,2,2-trifluoro-ethanone: 0.2%).

### Step-II: Purification of Crude 1-(5,8,14-Triazatetracyclo[10.3.1.0^{2,11}.0^{4,9}]hexadeca-2(11),3,5,7,9-pentaene)-2,2,2-trifluoro-ethanone

The crude 1-(5,8,14-triazatetracyclo[10.3.1.0^{2,11}.0^{4,9}]hexadeca-2(11),3,5,7,9-pentaene)-2,2,2-trifluoro-ethanone (obtained in step-I) was dissolved in isopropanol (266 ml) at reflux temperature and the clear solution was cooled to 25-30° C. The resulting suspension was stirred for 3 hours at 25-30° C. The separated solid was filtered and washed with isopropanol (2×38 ml) and then dried at 50-55° C. to produce 30 g of pure 1-(5,8,14-triazatetracyclo[10.3.1.0^{2,11}.0^{4,9}]hexadeca-2(11),3,5,7,9-pentaene)-2,2,2-trifluoro-ethanone (Purity by HPLC: 99.89%; Content of 1-(6-Methyl-5,8,14-triazatetracyclo[10.3.1.0^{2,11}.0^{4,9}]hexadeca-2(11),3,5,7,9-pentaene)-2,2,2-trifluoro-ethanone 0.10%).

### Step-III: Preparation of Varenicline Free Base

The pure 1-(5,8,14-triazatetracyclo[10.3.1.0^{2,11}.0^{4,9}]hexadeca-2(11),3,5,7,9-pentaene)-2,2,2-trifluoro-ethanone (100 g, obtained in step-II) was slurried in methanol (1000 ml), followed by treatment with sodium hydroxide (26 g) in water (50 ml). The mixture was heated for 1 hour at 40-45° C., concentrated under vacuum at below 45° C., followed by the addition of water (500 ml) and adjusting the pH to 10 with glacial acetic acid at 25-30° C. The resulting mass was extracted with methylene chloride (2×1000 ml) and the organic layer was washed with water (1000 ml) and then dried over anhydrous sodium sulfate. Methylene dichloride was distilled out under vacuum, followed by degassing for 30 minutes to give varenicline free base as a residue (Purity by HPLC: 99.75%; Content of Methylvarenicline impurity: 0.10%).

### Step-IV: Purification of Varenicline Free Base

The varenicline free base (obtained in step-III) was suspended in a mixture of methanol and diisopropyl ether (10:90; methanol:diisopropyl ether; 300 ml), the suspension was heated at 55-60° C. and then stirred for 1 hour at 55-60° C. The suspension was cooled to 25-30° C. and then stirred for 1 hour at 25-30° C. The resulting solid was filtered and washed with a mixture of methanol and diisopropyl ether (10:90, methanol:diisopropyl ether; 100 ml). The brownish color solid obtained was dried at 50-55° C. to give 50 g of pure varenicline (Purity by HPLC: 99.94%; Content of Methylvarenicline impurity: 0.03%).

### Example 2

### Preparation of 5,8,14-Triazatetracyclo[10.3.1.0^{2,11},0^{4,9}]hexadeca-2(11),3,5,7,9-pentaene, (2R,3R)-2,3-dihydroxybutanedioate (1:1) (Varenicline Tartrate)

Varenicline free base (50 g, obtained in step-IV of example 1) was dissolved in methanol (300 ml) and the solution was added to a solution of tartaric acid (39.31 g) dissolved in methanol (300 ml) at 20-25° C. The resulting suspension was stirred for 1 hour 30 minutes at 20-25° C. The separated solid was filtered and then dried to yield 80 g of varenicline tartrate (Purity by HPLC: 99.97%; Content of Methylvarenicline impurity: 0.02%).

### Example 3

### Preparation of 6-Methyl-5,8,14-Triazatetracyclo[10.3.1.0^{2,11}.0^{4,9}]hexadeca-2(11),3,5,7,9-pentaene (Methylvarenicline)

### Step-I: Preparation of 1-(6-Methyl-5,8,14-triazatetracyclo[10.3.1.0^{2,11}.0^{4,9}]hexadeca-2(11),3,5,7,9-pentaene)-2,2,2-trifluoro-ethanone

1-(4,5-Diamino-10-aza-tricyclo[6.3.1.0^{2,7}]dodeca-2(7),3,5-trien-10-yl)-2,2,2-trifluoro-ethanone (32 g), lead monoxide (25 g) and chloroacetone (5.18 g) were added to 20% aqueous dimethyl sulfoxide (80:20, dimethyl sulfoxide:water) (512 ml), and the mixture was heated at 60° C. The reaction mixture was stirred for 30 minutes at 60° C., followed by the addition of chloroacetone (2.59 g) and aqueous dimethylsulfoxide (32 ml) at 60° C. and then stirring the reaction mixture for 30 minutes at 60° C. To the resulting mass was added chloroacetone (2.59 g) and aqueous dimethylsulfoxide (32 ml) at 60° C., followed by stirring the mixture at 55-60° C. for 12 hours. The reaction mass was cooled to 25-30° C., followed by the addition of water (544 ml). The unreacted lead monoxide was filtered and washed with water (544 ml). The filtrate was extracted with methylene chloride (3×300 ml) at 25-30° C. All the organic layers were combined and washed with 1 N hydrochloric acid (3×300 ml). The organic layer was dried over sodium sulfate and charcoalized. The methylene chloride layer was concentrated under vacuum at below 45° C., followed by recrystallization in isopropanol to yield 8.5 g of 1-(6-methyl-5,8,14-triazatetracyclo[10.3.1.0^{2,11}.0^{4,9}]hexadeca-2(11),3,5,7,9-pentaene)-2,2,2-trifluoro-ethanone (Purity by HPLC: 98.2%).

### Step-II: Preparation of 6-Methyl-5,8,14-triazatetracyclo[10.3.1.0^{2,11}.0^{4,9}]hexadeca-2(11),3,5,7,9-pentaene (Methylvarenicline)

1-(6-Methyl-5,8,14-triazatetracyclo[10.3.1.0^{2,11}.0^{4,9}]hexadeca-2(11),3,5,7,9-pentaene)-2,2,2-trifluoro-ethanone (5 g, obtained in step-I) was slurried in methanol (50 ml), followed by treatment with sodium hydroxide (1.28 g) in water (25 ml). The mixture was heated at 40-45° C. for 1 hour and then concentrated under vacuum at below 45° C., followed by treatment with water (50 ml) and adjusting the pH to 10 with glacial acetic acid at 25-30° C. The resulting mass was extracted with dichloromethane (2×50 ml), the organic layer was washed with water (50 ml) and then dried over anhydrous sodium sulfate. Dichloromethane was distilled out under vacuum, followed by crystallization in diisopropyl ether. The brownish color solid obtained was dried at 50-55° C. to give 2.5 g of 6-methyl-5,8,14-triazatetracyclo[10.3.1.0^{2,11}.0^{4,9}]hexadeca-2(11),3,5,7,9-pentaene (Purity by HPLC: 98.77%).

Unless otherwise indicated, the following definitions are set forth to illustrate and define the meaning and scope of the various terms used to describe the invention herein.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP).

As used herein, the term, “detectable” refers to a measurable quantity measured using an HPLC method having a detection limit of 0.01 area-%.

As used herein, in connection with amount of impurities in varenicline or a pharmaceutically acceptable salt thereof, the term “not detectable” means not detected by the herein described HPLC method having a detection limit for impurities of 0.01 area-%.

As used herein, “limit of detection (LOD)” refers to the lowest concentration of analyte that can be clearly detected above the base line signal, is estimated is three times the signal to noise ratio.

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (PSD)” means the cumulative volume size distribution of equivalent spherical diameters as determined by laser diffraction in Malvern Master Sizer 2000 equipment or its equivalent. “Mean particle size distribution, i.e., (D₅₀)” correspondingly, means the median of said particle size distribution.

The important characteristics of the PSD are the (D₉₀), which is the size, in microns, below which 90% of the particles by volume are found, and the (D₅₀), which is the size, in microns, below which 50% of the particles by volume are found. Thus, a D₉₀ or d(0.9) of less than 300 microns means that 90 volume-percent of the particles in a composition have a diameter less than 300 microns.

All ranges disclosed herein are inclusive and combinable. While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

1. Varenicline or a pharmaceutically acceptable salt thereof comprising a 6-methyl-5,8,14-triazatetracyclo[10.3.1.0^{2,11},0^{4,9}]hexadeca-2(11),3,5,7,9-pentaene impurity (methylvarenicline impurity) in an amount of less than 0.15 area-% as measured by HPLC.

2. Varenicline of claim 1, comprising the methylvarenicline impurity in an amount of about 0.01 area-% to about 0.1 area-%; wherein the varenicline or a pharmaceutically acceptable salt thereof has a purity of about 99.5% to about 99.99% as measured by HPLC; and wherein the pharmaceutically acceptable salt of varenicline is a hydrochloride salt, a hydrobromide salt, a sulphate salt, a phosphate salt, a tartrate salt, a fumarate salt, a maleate salt, an oxalate salt, an acetate salt, a propionate salt, a succinate salt, a mandelate salt, a mesylate salt, a besylate salt, or a tosylate salt.

3. Varenicline of claim 2, comprising the methylvarenicline impurity in an amount of about 0.01 area-% to about 0.05 area-%; and wherein the pharmaceutically acceptable salt of varenicline is a tartrate salt.

4. Varenicline of claim 1, having a non-detectable amount of the methylvarenicline impurity as measured by HPLC.

5. A process for preparing the highly pure varenicline or a pharmaceutically acceptable salt thereof of claim 1, comprising:
a) reacting a protected diaminoazatricyclo compound of formula III: 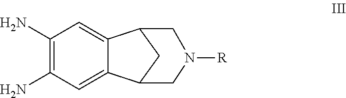
wherein ‘R’ represents a nitrogen protecting group, with a haloacetaldehyde compound of formula IV: 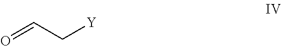
wherein ‘Y’ represents a halogen atom selected from the group consisting of F, Cl, Br and I; optionally in the presence of an oxygen source, to produce a crude protected triazatetracyclo compound of formula II: 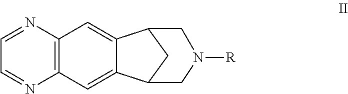
wherein R is as defined in formula III; and
b) recrystallizing the crude protected triazatetracyclo compound of formula II obtained in step-(a) from a first solvent to produce a pure protected triazatetracyclo compound of formula II;
c) deprotecting the pure compound of formula II obtained in step-(b) to produce a reaction mass containing varenicline free base;
d) recovering the varenicline free base as a residue from the reaction mass obtained in step-(c);
e) dissolving or suspending the varenicline free base obtained in step-(d) in a solvent medium comprising a second solvent and a third solvent to produce a solution or suspension, wherein the second solvent is an alcohol solvent and wherein the third solvent is an ether solvent; and
f) isolating and/or recovering highly pure varenicline free base substantially free of methylvarenicline impurity from the solution or suspension obtained in step-(e), and optionally converting the varenicline obtained in to a pharmaceutically acceptable salt thereof.

6. The process of claim 5, wherein the reaction in step-(a) is carried out in the presence of a solvent selected from the group consisting of water, an alcohol, a chlorinated hydrocarbon, a ketone, a polar aprotic solvent, a nitrile, an ester, and mixtures thereof; wherein the oxygen source employed in step-(a) is selected from the group consisting of lead monoxide, manganese dioxide, mercuric iodide and ceric ammonium nitrate; wherein the first solvent used in step-(b) is selected from the group consisting of water, an alcohol, a ketone, a nitrile, and mixtures thereof; wherein the second solvent used in step-(e) is selected from the group consisting of methanol, ethanol, n-propanol, isopropanol, n-butanol, isobutanol, tert-butanol, amyl alcohol, and mixtures thereof; and wherein the third solvent used in step-(e) is selected from the group consisting of tetrahydrofuran, 2-methyltetrahydrofuran, dioxane, diethyl ether, diisopropyl ether, monoglyme, diglyme, and mixtures thereof.

7. The process of claim 6, wherein the reaction in step-(a) is carried out in the presence of a solvent selected from the group consisting of water, dimethylsulfoxide, N,N-dimethylformamide, dimethylacetamide, and mixtures thereof; wherein the oxygen source employed in step-(a) is lead monoxide; wherein the first solvent used in step-(b) is selected from the group consisting of water, methanol, ethanol, isopropanol, n-butanol, and mixtures thereof; wherein the second solvent used in step-(e) is methanol; and wherein the third solvent used in step-(e) is diisopropyl ether.

8. The process of claim 5, wherein the condensation reaction in step-(a) is carried out at a temperature of about 0° C. to the reflux temperature of the solvent used; wherein the nitrogen protecting group ‘R’ in the compounds of formulae II and III is selected from the group consisting of acetyl, trifluoroacetyl, trichloroacetyl, pyrrolidinylmethyl, cumyl, benzhydryl, trityl, benzyloxycarbonyl (Cbz), 9-fluorenylmethyloxy carbonyl (Fmoc), benzyloxymethyl (BOM), pivaloyloxymethyl (POM), trichloroethxoycarbonyl (Troc), 1-adamantyloxycarbonyl (Adoc), allyl, allyloxycarbonyl, trimethylsilyl, tert.-butyldimethylsilyl, triethylsilyl (TES), triisopropylsilyl, trimethylsilylethoxymethyl (SEM), t-butoxycarbonyl (BOC), t-butyl, 1-methyl-1,1-dimethylbenzyl, pyrridinyl and pivaloyl; wherein the halogen atom ‘Y’ in the compound of formula IV is Cl; wherein the recrystallization in step-(b) is carried out by dissolving the crude triazatetracyclo compound of formula II in the first solvent to form a clear solution, and crystallizing the pure triazatetracyclo compound of formula II from the solution by forcible or spontaneous crystallization; wherein the deprotection in step-(c) is carried out by treating the protected triazatetracyclo compound of formula II with a base in a reaction inert solvent; wherein the varenicline free base in step-(e) is dissolved in the solvent medium at a temperature of about 30° C. to about the reflux temperature of the solvent medium used; wherein the suspension in step-(e) is prepared by suspending the varenicline free base in the solvent medium while stirring at a temperature of about 0° C. to about the reflux temperature of the solvent medium used; wherein the solution obtained in step-(e) is optionally subjected to carbon treatment or silica gel treatment; wherein the isolation in step-(f) is carried out by cooling the solution or suspension while stirring at a temperature of below 30° C.; and wherein the recovering in steps-(d) and (f) is, each independently, accomplished by filtration, filtration under vacuum, decantation, centrifugation, filtration employing a filtration media of a silica gel or celite, or a combination thereof.

9. The process of claim 8, wherein the condensation reaction in step-(a) is carried out at the reflux temperature of the solvent used; wherein the nitrogen protecting group ‘R’ is trifluoroacetyl; wherein the crystallization in step-(b) is carried out by cooling the solution while stirring at a temperature of about 0° C. to about 30° C.; wherein the varenicline free base in step-(e) is dissolved in the solvent medium at the reflux temperature of the solvent medium used; wherein the suspension obtained in step-(e) is stirred at a temperature of about 45° C. to about 80° C. for about 1 hour to about 10 hours; and wherein the isolation in step-(f) is carried out by cooling the solution or suspension while stirring at a temperature of about 0° C. to about 30° C.

10. A methylvarenicline compound, 6-methyl-5,8,14-triazatetracyclo[10.3.1.0^{2,11},0^{4,9}]hexadeca-2(11),3,5,7,9-pentaene, of formula A: 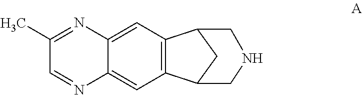 [SMILES: Cc1cnc2cc3c(cc2n1)C1CNCC3C1]
or a pharmaceutically acceptable acid addition salt thereof.

11. A process for synthesizing and isolating the methylvarenicline compound or a pharmaceutically acceptable acid addition salt thereof of claim 10, comprising:
a) reacting a protected diaminoazatricyclo compound of formula III: 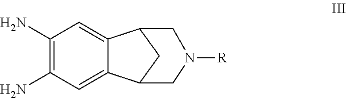
wherein ‘R’ represents a nitrogen protecting group, with a haloacetone compound of formula VI: 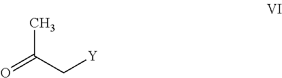
wherein ‘Y’ represents a halogen atom selected from the group consisting of F, Cl, Br and I; optionally in the presence of an oxygen source, to produce a protected triazatetracyclo compound of formula V: 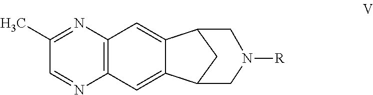
wherein R is as defined in formula III; and
b) deprotecting the compound of formula V to produce a reaction mass containing methylvarenicline of formula A; and
c) isolating and/or recovering methylvarenicline of formula A from the reaction mass obtained in step-(b) and optionally converting the methylvarenicline obtained in to a pharmaceutically acceptable acid addition salt thereof.

12. The process of claim 11, wherein the reaction in step-(a) is carried out in the presence of a solvent selected from the group consisting of water, methanol, ethanol, isopropyl alcohol, n-propanol, tert-butanol, n-butanol, methylene chloride, ethyl dichloride, chloroform, carbon tetrachloride, acetone, methyl isobutyl ketone, N,N-dimethylformamide, N,N-dimethylacetamide, dimethylsulfoxide, acetonitrile, propionitrile, ethyl acetate, isopropyl acetate, and mixtures thereof; wherein the oxygen source employed in step-(a) is selected from the group consisting of lead monoxide, manganese dioxide, mercuric iodide and ceric ammonium nitrate; wherein the nitrogen protecting group ‘R’ in the compounds of formulae III and V is selected from the group consisting of acetyl, trifluoroacetyl, trichloroacetyl, pyrrolidinylmethyl, cumyl, benzhydryl, trityl, benzyloxycarbonyl (Cbz), 9-fluorenylmethyloxy carbonyl (Fmoc), benzyloxymethyl (BOM), pivaloyloxymethyl (POM), trichloroethxoycarbonyl (Troc), 1-adamantyloxycarbonyl (Adoc), allyl, allyloxycarbonyl, trimethylsilyl, tert.-butyldimethylsilyl, triethylsilyl (TES), triisopropylsilyl, trimethylsilylethoxymethyl (SEM), t-butoxycarbonyl (BOC), t-butyl, 1-methyl-1,1-dimethylbenzyl, pyrridinyl and pivaloyl; wherein the halogen atom ‘Y’ in the compound of formula VI is Cl; wherein the deprotection in step-(b) is carried out by treating the protected triazatetracyclo compound of formula V with a base in a reaction inert solvent; and wherein the isolation of methylvarenicline in step-(c) is carried out using a solvent by cooling, seeding, partial removal of the solvent from the solution, by adding an anti-solvent to the solution, evaporation, vacuum distillation, or a combination thereof.

13. The process of claim 12, wherein the solvent used in step-(a) is selected from the group consisting of water, dimethylsulfoxide, N,N-dimethylformamide, dimethylacetamide, and mixtures thereof; wherein the oxygen source employed in step-(a) is lead monoxide; wherein the nitrogen protecting group ‘R’ is trifluoroacetyl; wherein the base used for deprotection in step-(b) is selected from the group consisting of ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide; wherein the solvent used for isolating the methylvarenicline in step-(c) is selected from the group consisting of water, methanol, ethanol, isopropyl alcohol, t-butanol, acetone, dichloromethane, tetrahydrofuran, dioxane, diethyl ether, diisopropyl ether, monoglyme, diglyme, and mixtures thereof; and wherein the isolation in step-(c) is carried out by cooling the solution while stirring at a temperature of about 0° C. to about 30° C.

14. A pharmaceutical composition comprising the highly pure varenicline or a pharmaceutically acceptable salt thereof of claim 1, and one or more pharmaceutically acceptable excipients.

15. The pharmaceutical composition of claim 14, wherein the varenicline or a pharmaceutically acceptable salt thereof has a D₉₀ particle size of less than or equal to about 300 microns.

16. The pharmaceutical composition of claim 15, wherein the D₉₀ particle size is about 1 micron to about 200 microns.
